(19) United States
(12) Patent Application Publication
(10) Pub. No.: US 2005/0214762 A1
(43) Pub. Date: Sep. 29, 2005

(54) Polypeptide variants

(75) Inventors: Richard Ross, Sheffield (GB); Peter Artymiuk, Sheffield (GB); Jon Sayers, Sheffield (GB)

Correspondence Address: CROWELL & MORING LLP;INTELLECTUAL PROPERTY GROUP, P.O. BOX 14300, WASHINGTON, DC, 20044-4300, US

(73) Assignee: ASTERION LIMITED, Sheffield (GB)

(21) Appl. No.: 10/502,344

(22) PCT Filed: Jan. 24, 2003
(86) PCT No.: PCT/GB03/00253

§ 371 Date: May 11, 2005

(30) Foreign Application Priority Data
Feb. 25, 2002 (GB) 0201679.8

(51) Int. Cl. C12Q 1/68; C07H 21/04; C12N 15/09; C07K 14/715

(52) U.S. Cl. 435/6; 435/69.1; 435/320.1; 435/325; 530/350; 536/23.2

(57) ABSTRACT

The invention relates to the provision of oligomeric polypeptides (dimers, trimmers, etc) comprising the ligand binding domains of cytokines which are linked via flexible polypeptides linker molecules. The linker molecules optionally comprise protease sensitive sites to modulate the release of biologically active cytokines when administered to a human or animal subject.

The invention relates to polypeptides which comprise more than two ligand binding domains of a cytokine wherein the domains are linked by a flexible linker which optionally comprises a proteolytic cleavage site.

Ligands which interact with receptors to bring about a suitable biochemical response are known as agonists and those that prevent, or hinder, a biochemical response are known as antagonists. For example, and not by way of limitation, cell specific growth factors are ligands that act as agonists and bind receptors located at the cell surface. Activation of the receptors by ligand-specific binding promotes cell proliferation via activation of intracellular signalling cascades that result in the expression of, amongst other things, cell-cycle specific genes, and the activation of quiescent cells to proliferate. Growth factors may also activate cellular differentiation.

A large group of growth factors, referred to as cytokines, are involved in a number of diverse cellular functions. These include, by example and not by way of limitation, modulation of the immune system, regulation of energy metabolism and control of growth and development. Cytokines which are secreted by lymphocytes are termed lymphokines (also known as interleukins). Those secreted by monocytes and macrophages are termed monokines. Cytokines are also secreted by endocrine glands, (for example growth hormone (GH) by the pituitary gland), and adipose cells (for example leptin). Cytokines mediate their effects via receptors expressed at the cell surface of target cells.

Receptors of the cytokine receptor family possess a single transmembrane domain and lack intrinsic enzyme activity (Kishimoto et al., 1994). Upon binding of a cytokine to a cognate receptor, either receptor homo- or hetero-dimerisation or oligomerisation occurs. The receptor complex is internalised and signalling occurs through the activation of associated signalling cascades that include the Jak/Stat and Mapk pathways. Internalisation is followed by a recycling step whereby the receptor molecule is regenerated for further use within the cell.

An example of the above is described with respect to GH and its binding to the GHR. This example is merely meant to be illustrative and not limiting and is an example of a cytokine which activates a signal transduction cascade by binding, dimerisation and internalisation of the receptor:ligand complex. It is known that a single molecule of growth hormone (GH) associates with two identical receptor molecules (Cunningham et al., 1991; de Vos et al., 1992; Sundstrom et al., 1996; Clackson et al., 1998). This occurs through two unique receptor-binding sites on GH and a common binding pocket on the extracellular domain of two receptors. Site 1 on the GH molecule has a higher affinity than site 2, and receptor dimerization is thought to occur sequentially with one receptor binding to site 1 on GH followed by recruitment of a second receptor to site 2.

The extracellular portion of the GHR exists as two linked domains each of approximately 100 amino acids (SD-100), the C-terminal SD-100 domain being closest to the cell surface and the N-terminal SD-100 domain being furthest away. It is a conformational change in these two domains that occurs on hormone binding with the formation of the hetero-trimeric complex GHR-GH-GHR. It has been proposed that ligand-driven receptor dimerization is the key event leading to signal activation (Cunningham et al., 1991), triggering phosphorylation cascades that include the Jak2/Stat5 pathway (Argetsinger & Carter-Su, 1996). Using confocal microscopy and Fluorescence Resonance Energy Transfer (FRET) it is known that there is very rapid internalisation of GHR after ligand binding and that internalisation and signaling are independent functions (Maamra et al., 1999). Internalisation of the GHR-GH-GHR complex is followed by a recycling step whereby the receptor molecule is regenerated for further use within the cell.

Examples of cytokines which are related to GH are leptin and erythropoietin (EPO). The leptin receptor and the EPO receptor share considerable structural homology with the GHR and require a similar dimerisation process to trigger signalling. Leptin supresses appetite and leptin resistance is associated with obesity. A leptin receptor antagonist will provide a treatment for anorexia nervosa. EPO excess causes polycythaemia which may be secondary to hypoxia (chronic lung disease), or primary in the case of polycythaemia rubra vera (a disorder of excess red blood cells). An EPO antagonist will provide a therapy for polycythaemia.

The disorders of acromegaly and gigantism result from an excess of growth hormone, usually due to pituitary tumours. A drug currently under trial is the pegylated GH antagonist B2036, designed using recently acquired knowledge of the molecular structure of the GHR. Unfortunately, to antagonise GH action very high levels of B2036 are required, over a 1000 times higher than endogenous GH levels.

The invention relates, inter alia, to the provision of oligomeric polypeptides (dimers, trimers etc) comprising the ligand binding domains of cytokines which are linked via flexible polypeptide linker molecules which optionally include protease sensitive sites to modulate the release of biologically active cytokines when administered to an animal.

According to a first aspect of the invention there is provided a polypeptide comprising more than two ligand binding domains of a cytokine receptor wherein said domains are linked by a linker molecule.

Preferably the linker molecule comprises at least one proteolytic cleavage site.

Preferably said cleavage site is sensitive to a serum protease.

Preferably said cleavage site comprises the amino acid sequence: LVPRGS (SEQ ID:1), or variant thereof.

In a further preferred embodiment of the invention said cleavage site comprises at least one copy of the amino acid sequence: SGGGG (SEQ ID:2), or functional variant thereof. Preferably, said cleavage site comprises the amino acid sequence PGISGGGGGG (SEQ ID:3).

More preferably still said cleavage site comprises the amino acid sequence: LVPRGSPGISGGGGGG (SEQ ID:4), or variant thereof.

Alternatively, said cleavage site comprises at least two copies of the amino acid sequence SGGGG, or functional variant thereof, which flank said cleavage site.

In a further preferred embodiment of the invention said cleavage site is sensitive to the serum protease thrombin.

In a further preferred embodiment of the invention said polypeptide comprises a plurality of ligand binding domains. Preferably said polypeptide has 3, 4, 5, 6, 7, 8, 9, or 10 ligand binding domains. Preferably said polypeptide has greater than 10 ligand binding domains.

In a further preferred embodiment of the invention said polypeptide has 4, 6, 8, 10, or 12 ligand binding domains.

In a preferred embodiment of the invention said polypeptide comprises at least four ligand binding domains.

In a further embodiment of the invention said polypeptide is an antagonist.

In an alternative preferred embodiment of the invention said polypeptide is an agonist.

In a further preferred embodiment of the invention said ligand binding domain is selected from the ligand binding domains of the cytokines selected from the group consisting of: growth hormone; leptin; erythropoietin; prolactin; interleukins (IL), IL-2, IL-3, IL-4, IL-5, IL-6, IL-7, IL-9, IL-10, IL-11; the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukaemia inhibitory factor (LIF); oncostatin M (OSM); interferon, IFNα and IFNγ.

In a preferred embodiment of the invention said ligand binding domain is the ligand binding domain of growth hormone.

A single hGH molecule binds to two GHR molecules. The hGH molecule interacts with one receptor molecule through a high affinity site, and with the other through a low affinity site. A single protein chain consisting of hGH linked to hGH will contain two high affinity sites which can bind strongly to a pair of receptor molecules and two low affinity sites.

In one embodiment of the invention, two or more copies of a ligand binding domain are expressed on a single polypeptide chain and the sequence of the tandem or oligomeric cytokine is arranged ‘ligand binding domain-linker-ligand binding domain’.

In a further preferred embodiment of the invention said ligand binding domain is the binding domain of leptin.

Preferably the linker comprises at least one copy of the peptide:

Gly Gly Gly Gly Ser Ser Ser Ser (hereinafter referred to as “Gly4Ser4”) (SEQ ID:5).

In one embodiment of the invention the linker is 8 amino acids in length and consists of one copy of the Gly4Ser4 linker. In an alternative embodiment of the invention, the linker is 16 amino acids in length and consists of two copies of the Gly4Ser4 linker. In a further alternative embodiment of the invention said linker is 24 amino acids in length and consists of three copies of the Gly4Ser4 linker.

More preferably the linker is a polypeptide which comprises 5 to 50 amino acid residues. Most preferably the linker comprises 5 to 30 amino acid residues.

Most preferably the linker comprises at least one copy of the peptide:

Gly Gly Gly Gly Ser (hereinafter referred to as “(Gly4Ser)” or (G₄S)) (SEQ ID:6).

In one embodiment of the invention the linker is 5 amino acids in length and consists of one copy of the (Gly4Ser) linker. In an alternative embodiment of the invention, the linker is 10 amino acids in length and consists of two copies of the (Gly4Ser)2 linker. In a further alternative embodiment of the invention said linker is 15 amino acids in length and consists of three copies of the (Gly4Ser) linker. In a further alternative embodiment of the invention said linker is 20 amino acids in length and consists of 4 copies of the (Gly4Ser)4 linker.

In an alternative embodiment of the invention, the polypeptide is a fusion protein comprising inframe translational fusions of ligand binding domains according to the invention.

It will be apparent to one skilled in the art that alternative linkers can be used to link ligand binding domains, for example the use of chemical protein crosslinkers. For example homo-bifunctional crosslinker such as disuccinimidyl-suberimidate-dihydrochloride; dimethyl-adipimidate-dihydrochloride; 1,5,-2,4 dinitrobenezene or hetero-bifunctional crosslinkers such as N-hydroxysuccinimidyl 2, 3-dibromopropionate; 1-ethyl-3-[3-dimethylaminopropyl] carbodiimide hydrochloride; succinimidyl 4-[n-maleimidomethyl]-cyclohexane-1-carboxylate.

Further examples of chemical crosslinks include the provision of chemically modified linker molecules and/or ligand binding domains. For example, if one end of the linker molecule is terminated with an amino terminal lysine residue and the ligand binding with a carboxyl-terminal glutamine residue then ligand binding domains can be oligomerised with transglutaminase.

According to a further aspect of the invention there is provided a nucleic acid molecule comprising a nucleic acid sequence which encodes a polypeptide according to the invention.

In a preferred embodiment of the invention said nucleic acid molecule comprises a nucleic acid sequence selected from the group consisting of:

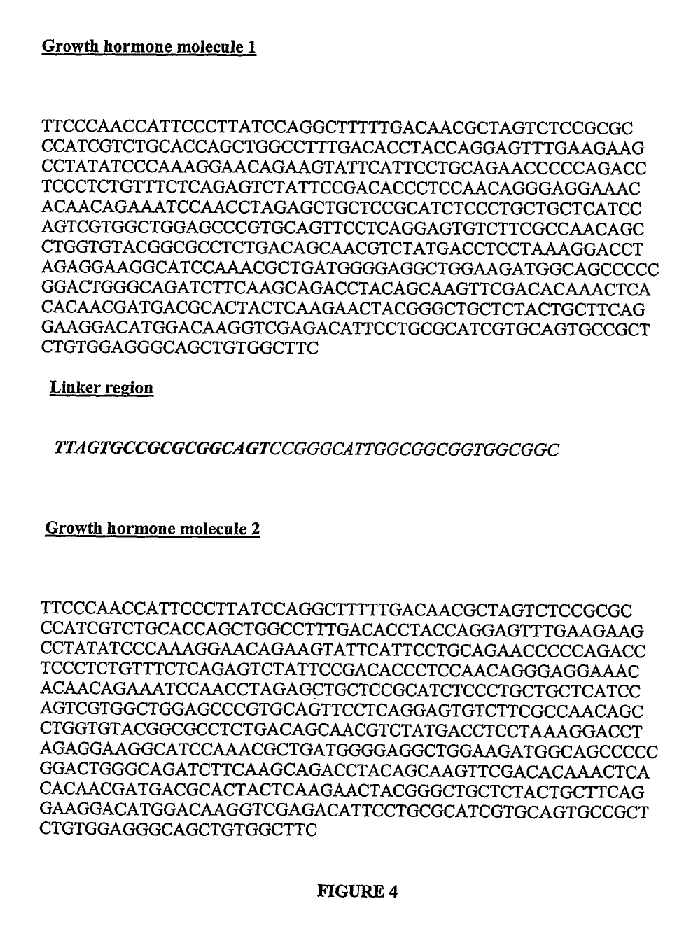

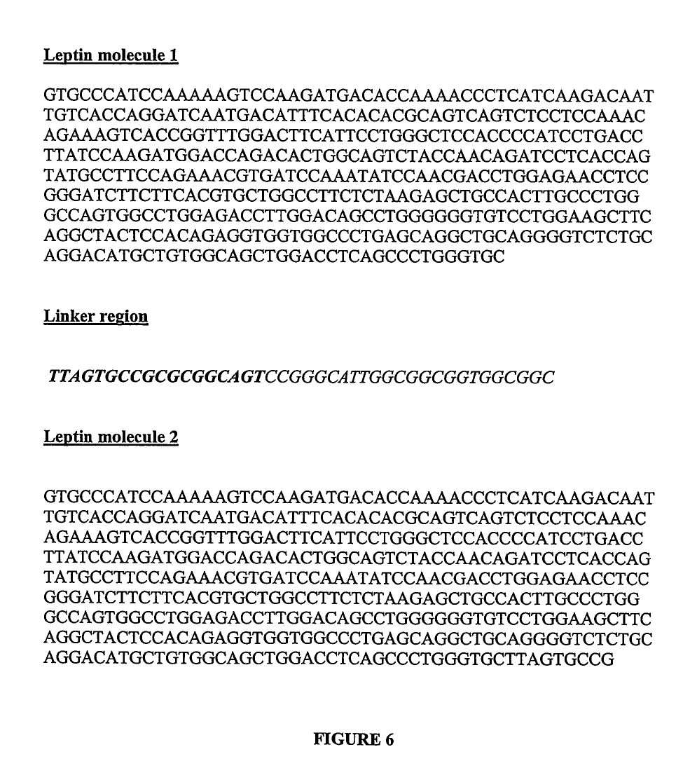

i) a sequence represented by FIGS. 4 or 6;

ii) a sequence which hybridises to the sequence of (i) above and which has cytokine receptor modulating activity; and

iii) a sequence which is degenerate as a result of the genetic code to the sequences defined in (i) and (ii) above.

In a preferred embodiment of the invention said nucleic acid hybridises under stringent hybridisation conditions to the sequences represented in FIGS. 4 or 6.

It is well known in the art that optimal hybridisation conditions can be calculated if the sequence of the nucleic acid is known. For example, hybridisation conditions can be determined by the GC content of the nucleic acid subject to hybridisation. Please see Sambrook et al. (1989) Molecular Cloning; A Laboratory Approach. A common formula for calculating the stringency conditions required to achieve hybridisation between nucleic acid molecules of a specified homology is:
Tₘ=81.5° C.+16.6 Log[Na⁺]+0.41[% G+C]−0.63 (% formamide).

Typically, hybridisation conditions uses 4-6×SSPE (20×SSPE contains 175.3 g NaCl, 88.2 g NaH₂PO₄H₂O and 7.4 g EDTA dissolved to 1 litre and the pH adjusted to 7.4); 5-10× Denhardts solution (50× Denhardts solution contains 5 g Ficoll (type 400, Pharmacia), 5 g polyvinylpyrrolidone abd 5 g bovine serum albumen/500 ml; 100 μg-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 42°-65° C.

According to a further aspect of the invention there is provided a polypeptide which is encoded by a nucleic acid molecule according to the invention.

In a preferred embodiment of the invention the polypeptide so encoded is modified by deletion, addition or substitution of at least one amino acid residue. Ideally said modification enhances the antagonistic or agonistic effects of said polypeptide with respect to the inhibition or activation of receptor mediated cell signalling.

Alternatively, or preferably, said modification includes the use of modified amino acids in the production of recombinant or synthetic forms of polypeptides.

It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N6-trimethyllysine, cyclohexyalanine, D-amino acids, ornithine. The incorporation of modified amino acids may confer advantageous properties on polypeptides. For example, the incorporation of modified amino acids may increase the affinity of the polypeptide for its binding site, or the modified amino acids may confer increased in vivo stability on the polypeptide thus allowing a decrease in the effective amount of therapeutic polypeptide administered to a patient.

According to a yet further aspect of the invention there is provided a vector including a DNA molecule according to any preceding aspect or embodiment of the invention.

In a preferred embodiment of the invention said vector is provided with means to manufacture recombinantly the polypeptide of the invention.

In a preferred embodiment of the invention said vector is an expression vector adapted for prokaryotic gene expression.

Prokaryotic expression systems are well known in the art and comprise vectors adapted for high level constitutive and inducible expression. Inducible expression systems are particularly advantageous if the recombinant polypeptide is toxic to the bacterial cell. Induction of expression is tightly regulated by promoters responsive to various inducers (e.g. IPTG inducible). Bacterial cells can be grown to stationary phase before induction thereby reducing harmful effects of toxic polypeptides.

Additionally it is also well known in the art that certain polypeptides are difficult to manufacture recombinantly due, for example, to protein instability or problems of aggregation. It is well known that genetically modified bacterial strains are available which are mutated in genes (e.g. bacterial proteases) which facilitate the production of native and recombinant bacterial polypeptides.

In a further preferred embodiment of the invention said vector is an expression vector adapted for eukaryotic gene expression.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and are therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S. Latchman, Academic Press Ltd., San Diego) is responsive to a number of environmental cues which include, by example and not by way of limitation, intermediary metabolites (eg glucose, lipids), environmental effectors (eg light, heat).

Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors.

Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al. (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol. III IRL Press, Oxford UK; DNA Cloning: F M Ausubel et al., Current Protocols in Molecular Biology, John Wiley & Sons, Inc.(1994).

In yet a further aspect of the invention there is provided a method to prepare a polypeptide according to the invention comprising:

(i) growing a cell transformed or transfected with a nucleic acid or vector of the present invention in conditions conducive to the manufacture of said polypeptide; and

(ii) purifying said polypeptide from said cell, or its growth environment.

In a preferred method of the invention said vector encodes, and thus said recombinant polypeptide is provided with, a secretion signal to facilitate purification of said binding agent polypeptide.

In yet a further aspect of the invention there is provided a cell transformed/transfected with the vector or nucleic acid according to the invention.

Preferably said cell eukaryotic and is selected from: fungal; insect (e.g. Spodoptera frugiperda); amphibian; plant; mammalian.

More preferably said cell is prokaryotic and is an E. coli cell.

According to a further aspect of the invention there is provided the use of the polypeptide according to the invention as a pharmaceutical. Preferably there is provided a pharmaceutical composition comprising the polypeptide according to the invention. Preferably said pharmaceutical composition includes a carrier, excipient and/or a diluent.

In a further preferred embodiment of the present invention said polypeptide is used for the manufacture of a medicament for use in the treatment of a disease selected from the group consisting of: acromegaly; gigantism; GH deficiency; Turners syndrome; renal failure; osteoporosis; diabetes mellitus; cancer; obesity; insulin resistance; hyperlipidaemia; hypertension; anaemia; autoimmune and infectious disease; inflammatory disorders including rheumatoid arthritis.

The invention also provides for a method of treating a human or animal subject comprising administering an effective amount of the polypeptide, pharmaceutical composition or medicament to said subject.

The polypeptides or compositions of the invention can be administered by any conventional route, including injection or by gradual infusion over time. The administration may, for example, be oral, intravenous, intraperitoneal, intramuscular, intracavity, subcutaneous, transdermal or delivered by a non-pathological GMO engineered to secrete the polypeptide.

The pharmaceutical compositions used in the foregoing methods preferably are sterile and contain an effective amount of the polypeptide according to the invention for producing the desired response in a unit of weight or volume suitable for administration to a patient.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents (e.g., acetic acid in a salt; citric acid in a salt; boric acid in a salt; and phosphoric acid in a salt), preservatives (e.g., benzalkonium chloride; chlorobutanol; parabens and thimerosal, compatible carriers, and optionally other therapeutic agents.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of polypeptides which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provide a method of treatment comprising administering to an animal, preferably a human, an effective amount of the nucleic acid or vector according to the invention.

An embodiment of the invention will now be described by example only and with reference to the following figures wherein;

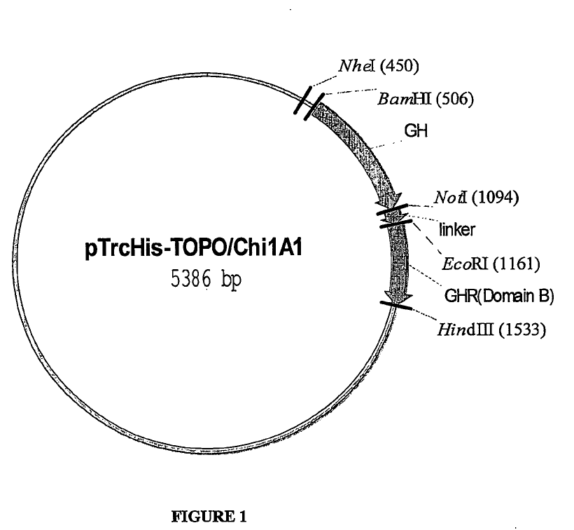

FIG. 1 illustrates a plasmid map of pTrcHisχ1A1.

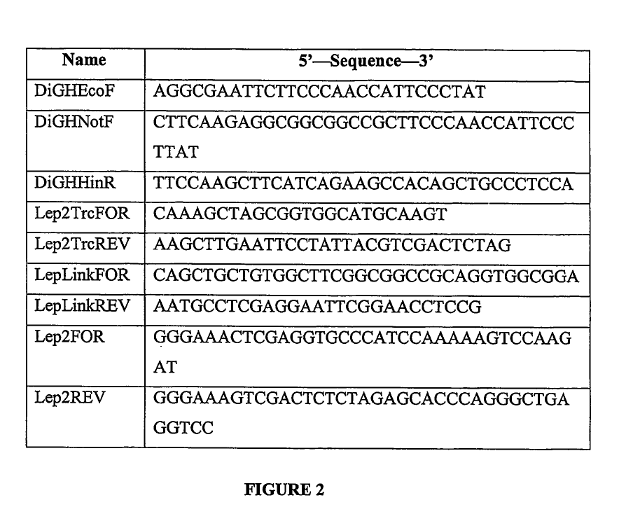

FIG. 2 illustrates primers used in the synthesis of the tandem constructs.

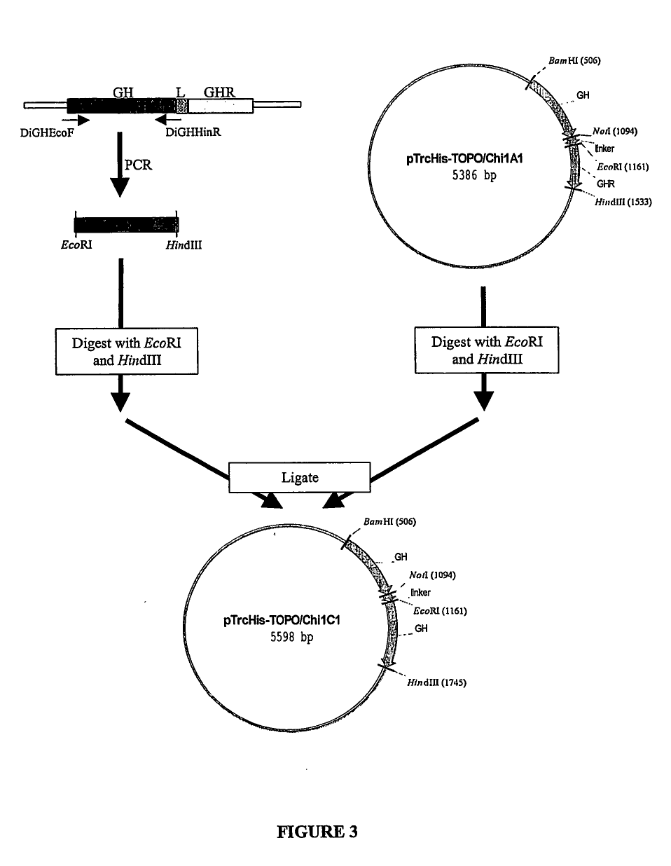

FIG. 3 is a schematic diagram for the construction of pTrcHisχ1C1. pTrcHisχ1C2 is constructed in the same way, however the forward primer used is DiGHNotF and the restriction enzymes used are NotI and HindIII.

FIG. 4 illustrates the DNA sequence for growth hormone tandem segregated by a thrombin cleavable linker. The linker region is shown in italics, with the thrombin cleavage site in bold.

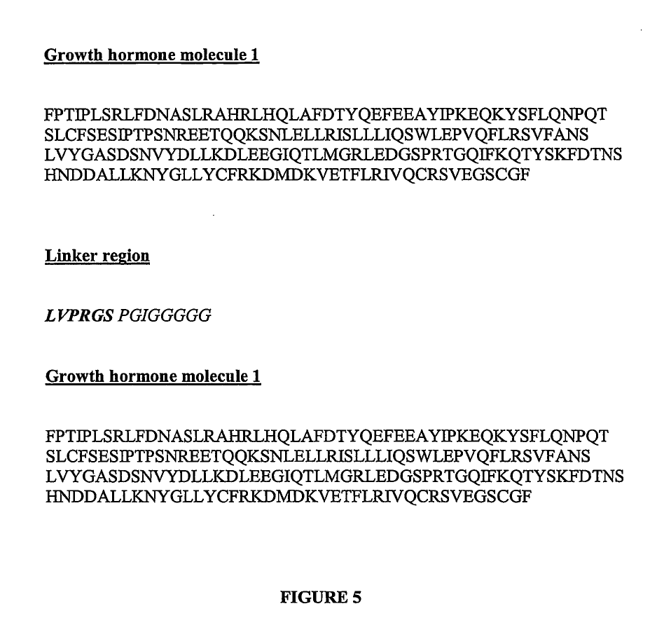

FIG. 5 illustrates the protein sequence for growth hormone tandem segregated by a thrombin cleavable linker. The linker region is shown in italics, with the thrombin cleavage site in bold.

FIG. 6 illustrates the DNA sequence for leptin tandem segregated by a thrombin cleavable linker. The linker region is shown in italics, with the thrombin cleavage site in bold.

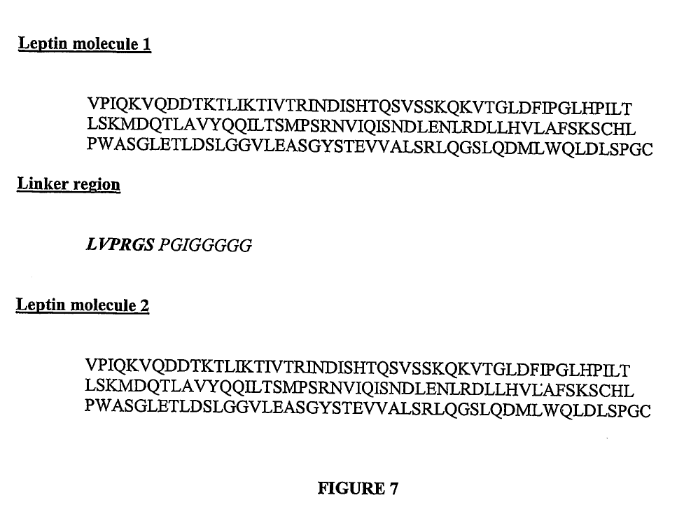

FIG. 7 illustrates the protein sequence for leptin tandem segregated by a thrombin cleavable linker. The linker region is shown in italics, with the thrombin cleavage site in bold. The cleavable linker is optional in this construct.

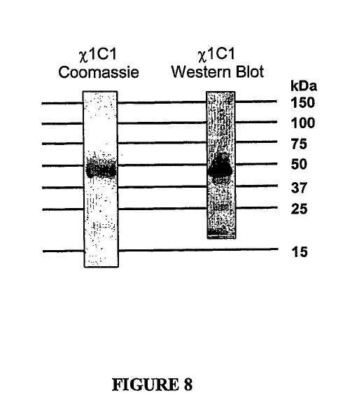

FIG. 8 shows the Coomassie stained SDS-PAGE gel and western blot of the purified GH-tandem (χ1C1).

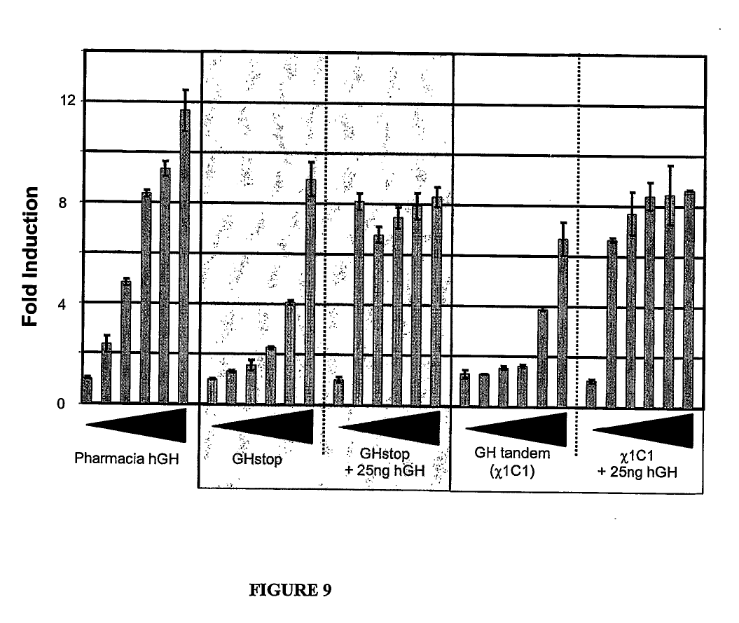

FIG. 9 is the bioassay data generated for χ1C1, with data for in-house synthesised GH and commercially produced GH for comparison. The activity of the tandem is similar to the in-house generated GH. In each case the concentrations of the test protein used were 0 ng, 6.25 ng, 12.5 ng, 25 ng, 50 ng and 100 ng.

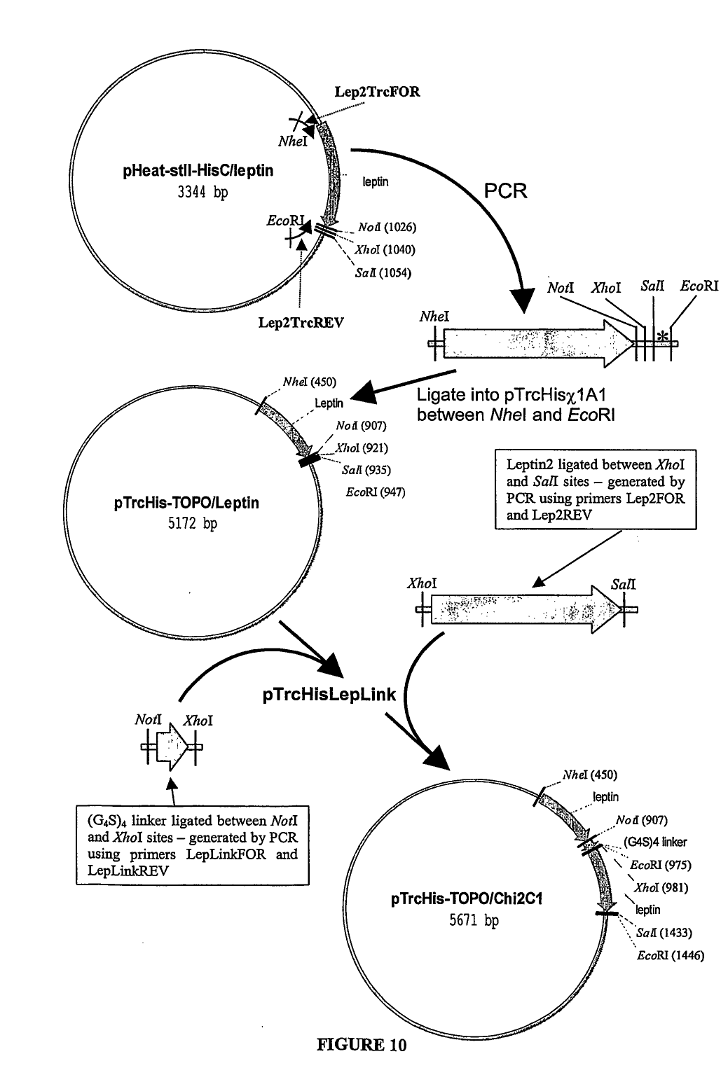

FIG. 10 is a schematic diagram illustrating the construction of pTrcHisχ2C1.

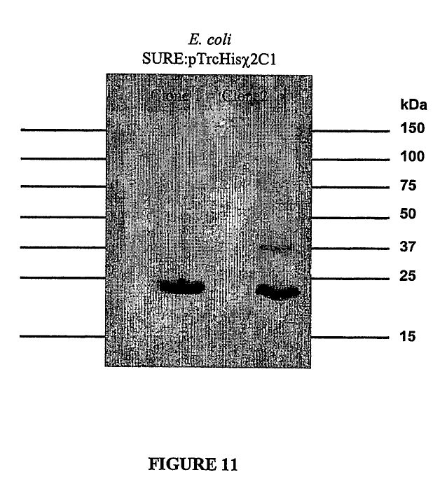

FIG. 11 shows the western blot of the SDS-PAGE gel on which the proteins expressed by clones of E. coli SURE:pTrcHisχ2C1 cells were run. The expected size of the leptin-tandem is ˜37 kDa and a faint band is observed at this size. The major band however is approximately half of this size. This suggests that the leptin tandem is being expressed but is most probably cleaved to the single leptin domains.

### MATERIALS AND METHODS

The sequence encoding GHR in pTrcHisχ1A1 (FIG. 1) was replaced with another GH gene (residues 1-191) to produce two successive GH genes linked with a (G₄S)₄ linker. The resultant construct (pTrcHisχ1C1) was transformed into E. coli SURE cells, a DNA recombination deficient strain of E. coli. Clones expressing GH-(G₄S)₄-GH protein were identified by western blotting using anti-GH (10A7, mouse IgG1) probed with Sheep anti-mouse-HRP (Amersham). Another GH-tandem, which lacked the (G₄S)₄ linker (GH-GH) was also constructed (pTrcHisχ1C2) using the same method.

GH-tandem protein was purified from cell lysates using a metal chelate affinity column (Probond resin, Invitrogen) followed by an ion exchange column (MonoQ, Pharmacia).

The effect of the GH-tandem proteins were analysed using an established bioassay (Ross et al., 1997).

The leptin gene was originally cloned into pHEAT; a temperature-inducible vector. However for expression in E. coli SURE cells the gene was sub-cloned into the pTrcHis plasmid. A (G₄S)₄ linker was then introduced and finally the second leptin domain was ligated into the gene to produce the construct that would express the leptin tandem. This construct pTrcHisχ2C1 was then transformed into E. coli SURE cells. Expression of the leptin-tandem was verified by western blot using anti-leptin antibodies (Sigma), developed in rabbit, probed with anti-rabbit-HRP (Sigma).

Cloning of the GH-Tandems

PCR was used to produce a fragment of DNA which consisted of a restriction site (NotI or EcoRI), followed by the GH gene and then a HindIII restriction site. This was ligated into pTrcHisχ1A1, which had been digested with the relevant restriction enzymes, to produce the constructs χ1C1 (GH-(G₄S)₄-GH) and χ1C2 (GH-GH). The primers used for the PCR's are shown in FIG. 2, and the reaction scheme is shown in FIG. 3.

Purification of the GH-Tandems

Induced cells (resuspended in 20 mM sodium phosphate buffer, 500 mM sodium chloride, pH 7.8) were lysed with a combination of 100 μg/ml (final concentration) hen egg white lysozyme and sonication. Insoluble material was removed by centrifugation at 400 rpm for 20 minutes.

The cleared cell lysate was applied to a 5 ml Probond resin column (Invitrogen), equilibrated with 20 mM sodium phosphate buffer, 500 mM sodium chloride, 5% glycerol, pH 7.8. The column was then washed with 10 column volumes of 20 mM sodium phosphate buffer, 500 mM sodium chloride, 5% glycerol, pH 6.0. Bound protein was eluted using 5 ml 20 mM sodium phosphate buffer, 500 mM sodium chloride, 5% glycerol, 500 mM imidazole, pH 6.0.

The protein was dialysed overnight against Low Salt Buffer (25 mM TRIS, 1 mM EDTA, 5% glycerol, pH 8.0) and then centrifuged to remove any particulate matter.

The protein sample was then loaded onto a Mono-Q column (Pharmacia), which had been pre-equilibrated with Low Salt Buffer. After a 10 column volume wash with Low Salt Buffer, the bound proteins were eluted over 20 column volumes using a gradient between 0M sodium chloride to 1M sodium chloride (in 25 mM TRIS, 1 mM EDTA, 5% glycerol, pH8.0). Peaks on the elution profile were analysed by SDS-PAGE and western blotting.

GH-tandem protein was then concentrated (if required) using a Amicon Centriprep Y-10 column.

The purity of the purified χ1C1 was confirmed by SDS-PAGE, by both coomassie staining and western blot (FIG. 8). Once the integrity of this sample had been confirmed, χ1C1 was submitted to the previously established bioassay (Ross et al., 1997) (FIG. 9).

Cloning of the Leptin-Tandems

PCR using the primers Lep2TrcFOR and Lep2TrcREV (FIG. 2) was used to generate DNA sequence consisting of (NheI)-LEPTIN-(NotI)-(XhoI)-(SalI)-STOP-(EcoRI) from pHEATLeptin. The terminal restriction sites were introduced by PCR and the internal restriction sites were already present in the pHEATLeptin vector. The PCR product generated was ligated into pTrcHisχ1A1 between NheI and EcoRI restriction sites to produce pTrcHisLeptin.

PCR was then used to generate the (G4S)₄-encoding linker flanked by NotI and XhoI restriction sites, the primers used were LepLinkFOR and LepLinkREV (FIG. 2). This was ligated into pTrcHisLeptin between the NotI and XhoI sites to produce pTrcHisLepLink.

The second leptin gene flanked by XhoI and SalI restriction sites was generated by PCR, using the primers Lep2FOR and Lep2REV (FIG. 2). This was ligated between the XhoI and SalI restriction sites to produce the construct which would express the leptin tandem, pTrcHisχ2C 1. This process for the generation of the leptin-tandem is shown in FIG. 10.

This plasmid was transformed into E. coli SURE cells and expression studies carried out, visualisation of expression was performed by western blot (FIG. 11)

References

ARGETSINGER, L. S. & CARTER-SU, C. (1996) Growth hormone signalling mechanisms: involvement of the tyrosine kinase JAK2. [Review] [19 refs]. Hormone Research, 45 Suppl 1, 22-24.
CHEN, C., BRINKWORTH, R. & WATERS, M. J. (1997) The role of receptor dimerization domain residues in growth hormone signaling. Journal of Biological Chemistry, 272, 5133-5140.
CHEN, W. Y., CHEN, N. Y., YUN, J., WAGNER, T. E. & KOPCHICK, J. J. (1994) In vitro and in vivo studies of antagonistic effects of human growth hormone analogs [published erratum appears in J Biol Chem 1994 Aug. 12;269(32):20806]. Journal of Biological Chemistry, 269, 15892-15897.
CHEN, W. Y., WHITE, M. E., WAGNER, T. E. & KOPCHICK, J. J. (1991) Functional antagonism between endogenous mouse growth hormone (GH) and a GH analog results in dwarf transgenic mice. Endocrinology, 129, 1402-1408.
CHEN, W. Y., WIGHT, D. C., MEHTA, B. V., WAGNER, T. E. & KOPCHICK, J. J. (1991) Glycine 119 of bovine growth hormone is critical for growth-promoting activity. Molecular Endocrinology, 5, 1845-1852.
CHEN, W. Y., WIGHT, D. C., WAGNER, T. E. & KOPCHICK, J. J. (1990) Expression of a mutated bovine growth hormone gene suppresses growth of transgenic mice. Proceedings of the National Academy of Sciences of the United States of America, 87, 5061-5065.
CLACKSON, T., ULTSCH, M. H., WELLS, J. A. & DE VOS, A. M. (1998) Structural and functional analysis of the 1:1 growth hormone:receptor complex reveals the molecular basis for receptor affinity. Journal of Molecular Biology, 277, 1111-1128.
CUNNINGHAM, B. C., ULTSCH, M., DE VOS, A. M., MULKERRIN, M. G., CLAUSER, K. R. & WELLS, J. A. (1991) Dimerization of the extracellular domain of the human growth hormone receptor by a single hormone molecule. Science, 254, 821-825.
DE VOS, A. M., ULTSCH, M. & KOSSIAKOFF, A. A. (1992) Human growth hormone and extracellular domain of its receptor: crystal structure of the complex. Science, 255, 306-312.
FUH, G., CUNNINGHAM, B. C., FUKUNAGA, R., NAGATA, S., GOEDDEL, D. V. & WELLS, J. A. (1992) Rational design of potent antagonists to the human growth hormone receptor. Science, 256, 1677-1680.
HANIU, M., ARAKAWA, T., BURES, E. J., YOUNG, Y., HUI, J. O., ROHDE, M. F., WELCHER, A. A. & HORAN, T. (1998) Human leptin receptor. Determination of disulfide structure and N-glycosylation sites of the extracellular domain. Journal of Biological Chemistry, 273, 28691-28699.
HIROIKE, T., HIGO, J., JINGAMI, H. & TOH, H. (2000) Homology modeling of human leptin/leptin receptor complex. Biochemical & Biophysical Research Communications, 275, 154-158.
HUKSHORN, C. J., SARIS, W. H., WESTERTERP-PLANTENGA, M. S., FARID, A. R., SMITH, F. J. & CAMPFIELD, L. A. (2000) Weekly subcutaneous pegylated recombinant native human leptin (PEG-OB) administration in obese men. [see comments].Journal of Clinical Endocrinology & Metabolism, 85, 4003-4009.
KISHIMOTO, T., TAGA, T. & AKIRA, S. (1994) Cytokine signal transduction. [Review] [92 refs]. Cell, 76, 253-262.
LIVNAH, O., STURA, E. A., MIDDLETON, S. A., JOHNSON, D. L., JOLLIFFE, L. K. & WILSON, I. A. (1999) Crystallographic evidence for preformed dimers of erythropoietin receptor before ligand activation. Science, 283, 987-990.
MAAMRA, M., FINIDORI, J., VON LAUE, S., SIMON, S., JUSTICE, S., WEBSTER, J., DOWER & ROSS, R. (1999) Studies with a growth hormone antagonist and dual-fluorescent confocal microscopy demonstrate that the full-length human growth hormone receptor, but not the truncated isoform, is very rapidly internalized independent of Jak2-Stat5 signaling. Journal of Biological Chemistry, 274, 14791-14798.
MELLADO, M., RODRIGUEZ-FRADE, J. M., KREMER, L., VON KOBBE, C., DE ANA, A. M., MERIDA, I. & MARTINEZ, A. (1997) Conformational changes required in the human growth hormone receptor for growth hormone signaling. Journal of Biological Chemistry, 272, 9189-9196.
ROSS, R. J., ESPOSITO, N., SHEN, X. Y., VON LAUE, S., CHEW, S. L., DOBSON, P. R., POSTEL-VINAY, M. C. & FINIDORI, J. (1997) A short isoform of the human growth hormone receptor functions as a dominant negative inhibitor of the full-length receptor and generates large amounts of binding protein. Molecular Endocrinology, 11, 265-273.
SUNDSTROM, M., LUNDQVIST, T., RODIN, J., GIEBEL, L. B., MILLIGAN, D. & NORSTEDT, G. (1996) Crystal structure of an antagonist mutant of human growth hormone, G120R, in complex with its receptor at 2.9 A resolution. Journal of Biological Chemistry, 271, 32197-32203.
SYED, R. S., REID, S. W., LI, C., CHEETHAM, J. C., AOKI, K. H., LIU, B., ZHAN, H., OSSLUND, T D, CHIRINO, A. J., ZHANG, J., FINER-MOORE, J., ELLIOTT, S., SITNEY, K., KATZ, B. A., MATTHEWS, D. J., WENDOLOSKI, J. J., EGRIE, J. & STROUD, R. M. (1998) Efficiency of signalling through cytokine receptors depends critically on receptor orientation. Nature, 395, 511-516.
THORNER, M. O., STRASBURGER, C. J., WU, Z., STRAUME, M., BIDLINGMAIER, M., PEZZOLI, S., ZIB, K., SCARLETT, J. C. & BENNETT, W. F. (1999) Growth hormone (GH) receptor blockade with a PEG-modified GH (B2036-PEG) lowers serum insulin-like growth factor-I but does not acutely stimulate serum GH. Journal of Clinical Endocrinology & Metabolism, 84, 2098-2103.

1. A polypeptide comprising more than two ligand binding domains of a cytokine receptor wherein said domains are linked by a linker molecule and wherein the linker molecule comprises at least one proteolytic cleavage site.

2. A polypeptide according to claim 1 wherein said cleavage site is sensitive to a serum protease.

3. A polypeptide according to claim 2 wherein the serum protease is thrombin.

4. A polypeptide according to claim 1 wherein said cleavage site comprises the amino acid sequence LVPRGS (SEQ ID: 1), or a variant thereof.

5. A polypeptide according to claim 1 wherein said cleavage site comprises the amino acid sequence SGGGG (SEQ ID:2), or a variant thereof.

6. A polypeptide according to claim 1 wherein said cleavage site comprises the amino acid sequence PGISGGGGGG (SEQ ID:3).

7. A polypeptide according to claim 4 wherein said cleavage site comprises the amino acid sequence: LVPRGSPGISGGGGGG (SEQ ID:4), or a variant thereof.

8. A polypeptide according to claim 5 wherein said cleavage site comprises a center and two copies of the amino acid sequence SGGGG, or a variant thereof, which flank the center of said cleavage site.

9. A polypeptide according to claim 1 wherein said polypeptide comprises at least four ligand binding domains.

10. A polypeptide according to claim 9 wherein said polypeptide comprises 4, 6, 8, 10, or 12 ligand binding domains.

11. A polypeptide according to claim 1 wherein said polypeptide comprises 3, 4, 5, 6, 7, 8, 9, or 10 ligand binding domains.

12. A polypeptide according to claim 9 wherein said polypeptide comprises greater than 10 ligand binding domains.

13. A polypeptide according to claim 1 wherein said polypeptide is an antagonist to said cytokine.

14. A polypeptide according to claim 1 wherein said polypeptide is an agonist to said cytokine.

15. A polypeptide according to claim 1 wherein said cytokine receptor ligand binding domain is the ligand binding domain of a cytokine selected from the group consisting of: growth hormone; leptin; erythropoietin; prolactin; interleukins (IL), IL-2, IL-3, IL-4, IL-5, IL-6, IL-7, IL-9, IL-10, IL-11, the p35 subunit of IL-12, IL-13, IL-15; granulocyte colony stimulating factor (G-CSF) granulocyte macrophage colony stimulating factor (GM-CSF); ciliary neurotrophic factor (CNTF), cardiotrophin-1 (CT-1), leukaemia inhibitory factor (LIF), oncostatin M (OSM), interferon, IFNα and IFNγ.

16. A polypeptide according to claim 15 wherein the binding domain is the ligand binding domain of growth hormone.

17. A polypeptide according to claim 16 wherein the binding domain is the ligand binding domain of leptin.

18. A polypeptide according to claim 1 wherein the linker is a polypeptide which comprises from 5 to 50 amino acid residues.

19. A polypeptide according to claim 18 wherein the linker comprises from 5 to 30 amino acid residues.

20. A polypeptide according to claim 1 wherein the linker comprises at least one copy of the peptide GGGGS.

21. A polypeptide according to claim 20 wherein the linker is 5 amino acids in length and consists of one copy of GGGGS (the Gly4Ser linker).

22. A polypeptide according to claim 20 wherein the linker is 10 amino acids in length and consists of two copies of the Gly4Ser linker.

23. A polypeptide according to claim 20 wherein the linker is 15 amino acids in length and consists of three copies of the Gly4Ser linker.

24. A polypeptide according to claim 20 wherein the linker is 20 amino acids in length and consists of 4 copies of the Gly4Ser linker.

25. A polypeptide according to claim 1 wherein the polypeptide is a fusion protein comprising inframe translational fusions of ligand binding domains.

26. A polypeptide according to claim 1 comprising chemical crosslinkers wherein the chemical crosslinkers serve to link the ligand binding domains.

27. A polypeptide according to claim 26 wherein the chemical crosslinker comprises a homo-bifunctional crosslinker selected from the group consisting of disuccinimidyl-suberimidate-dihydrochloride; dimethyl-adipimidate-dihydrochloride; and 1,5,-2,4 dinitrobenezene.

28. A polypeptide according to claim 26 or claim 27 wherein the crosslinker comprises a hetero-bifunctional crosslinker selected from the group consisting of N-hydroxysuccinimidyl 2, 3-dibromopropionate; 1-ethyl-3-[3-dimethylaminopropyl] carbodiimide hydrochloride; and succinimidyl 4-[n-maleimidomethyl]-cyclohexane-1-carboxylate.

29. A nucleic acid molecule comprising a nucleic acid sequence which encodes a polypeptide according to claim 1.

30. A nucleic acid molecule comprising the sequence selected from the group consisting of:
(i) the sequence represented by FIG. 4 or 6;
(ii) a sequence which hybridises to the sequence of (i) above and which has cytokine receptor modulating activity; and
(iii) a sequence which is degenerate as a result of the genetic code to the sequences defined in (i) and (ii) above.

31. A nucleic acid molecule which hybridises under stringent hybridisation conditions to the sequences represented in FIG. 4 or 6.

32. A polypeptide encoded by the nucleic acid molecule according to claim 29.

33. A polypeptide according to claim 32 wherein said polypeptide is modified by deletion, addition, and/or substitution of at least one amino acid residue and said modification enhances the antagonistic or agonistic effects of said polypeptide with respect to the inhibition or activation of receptor mediated cell signalling.

34. A vector comprising the nucleic acid molecule of claim 29.

35. A vector according to claim 34 wherein said vector is an expression vector adapted for prokaryotic or eukaryotic gene expression.

36. A vector according to claim 34 wherein said vector further encodes, a secretion signal linked to the polypeptide to facilitate purification of the polypeptide.

37. A method to prepare a polypeptide according to claim 1, the method comprising;
(i) growing a cell transformed or transfected with a nucleic acid of claim 29 in conditions conducive to the manufacture of said polypeptide; and
(ii) purifying said polypeptide from said cell, or its growth environment.

38. A cell transformed/transfected with the vector of the nucleic acid of claim 29.

39-42. (canceled)

43. A pharmaceutical composition comprising the polypeptide according to claim 1, and a pharmaceutically acceptable carrier, excipient, or a diluent.

44. A pharmaceutical composition comprising the nucleic acid molecule of claim 29 and a pharmaceutically acceptable excipient.

45. A method for treating a disease selected from the group consisting of: acromegaly; gigantism; GH deficiency; Turners syndrome; renal failure; osteoporosis; diabetes mellitus; cancer; obesity; insulin resistance; hyperlipidaemia; hypertension; anaemia; an autoimmune disease; an infectious disease; an inflammatory disorder, and rheumatoid arthritis, wherein said method comprising administering to a patient in need thereof a pharmaceutical composition according to claim 43.

46. A method for treating a disease selected from the group consisting of: acromegaly; gigantism; GH deficiency; Turners syndrome; renal failure; osteoporosis; diabetes mellitus; cancer; obesity; insulin resistance; hyperlipidaemia; hypertension; anaemia; an autoimmune disease; an infectious disease; an inflammatory disorder, and rheumatoid arthritis, wherein said method comprising administering to a patient in need thereof a pharmaceutical composition according to claim 44.


SEQUENCE LISTING (22 sequences)

SEQ ID NO: 1 - protein, 6 aa, Artificial
  Artificial linker
LVPRGS

SEQ ID NO: 2 - protein, 5 aa, Artificial
  Artificial linker
SGGGG

SEQ ID NO: 3 - protein, 10 aa, Artificial
  Artificial linker
PGISGGGGGG

SEQ ID NO: 4 - protein, 16 aa, Artificial
  Artificial linker
LVPRGSPGISGGGGGG

SEQ ID NO: 5 - protein, 8 aa, Artificial
  Artificial linker
GGGGSSSS

SEQ ID NO: 6 - protein, 5 aa, Artificial
  Artificial linker
GGGGS

SEQ ID NO: 7 - DNA, 28 bp, Artificial
  Artificial linker
aggcgaattcttcccaaccattccctat

SEQ ID NO: 8 - DNA, 39 bp, Artificial
  Artificial linker
cttcaagaggcggcggccgcttcccaaccattcccttat

SEQ ID NO: 9 - DNA, 35 bp, Artificial
  Artificial linker
ttccaagcttcatcagaagccacagctgccctcca

SEQ ID NO: 10 - DNA, 24 bp, Artificial
  Artificial linker
caaagctagcggtggcatgcaagt

SEQ ID NO: 11 - DNA, 30 bp, Artificial
  Artificial linker
aagcttgaattcctattacgtcgactctag

SEQ ID NO: 12 - DNA, 35 bp, Artificial
  Artificial linker
cagctgctgtggcttcggcggccgcaggtggcgga

SEQ ID NO: 13 - DNA, 27 bp, Artificial
  Artificial linker
aatgcctcgaggaattcggaacctccg

SEQ ID NO: 14 - DNA, 36 bp, Artificial
  Artificial linker
gggaaactcgaggtgcccatccaaaaagtccaagat

SEQ ID NO: 15 - DNA, 39 bp, Artificial
  Artificial linker
gggaaagtcgactctctagagcacccagggctgaggtcc

SEQ ID NO: 16 - DNA, 573 bp, Homo sapiens
ttcccaaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 17 - DNA, 42 bp, Homo sapiens
ttagtgccgcgcggcagtccgggcattggcggcggtggcggc

SEQ ID NO: 18 - protein, 191 aa, Artificial
  Artificial linker
FPTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 19 - protein, 14 aa, Artificial
  Artificial linker
LVPRGSPGIGGGGG

SEQ ID NO: 20 - DNA, 438 bp, Homo sapiens
gtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgc

SEQ ID NO: 21 - DNA, 447 bp, Homo sapiens
gtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcttagtgccg

SEQ ID NO: 22 - protein, 146 aa, Artificial
  Artificial linker
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC